(19) United States
(12) Patent Application Publication
(10) Pub. No.: US 2016/0317346 A1
(43) Pub. Date: Nov. 3, 2016

(54) SYSTEMS AND METHODS FOR MONITORING COOLING OF SKIN AND TISSUE TO IDENTIFY FREEZE EVENTS

(71) Applicant: Zeltiq Aesthetics, Inc., Pleasanton, CA (US)

(72) Inventor: Dennis Kovach, San Ramon, CA (US)

(21) Appl. No.: 15/140,415

(22) Filed: Apr. 27, 2016

Related U.S. Application Data
(60) Provisional application No. 62/153,896, filed on Apr. 28, 2015.

(51) Int. Cl. A61F 7/00

(52) U.S. Cl. CPC A61F 7/007; A61F 2007/0054; A61F 2007/0075; A61F 2007/0086

(57) ABSTRACT

A system and method of detecting, evaluating, monitoring events during the removal of heat from tissue beneath skin. The system utilizes an adaptive filter to determine if a partial freeze event is occurring, and performs an action based on the determination. In some examples, the system shuts off the treatment device, alerts an operator, reduces the cooling, and/or limits an amount of further cooling, in response to a determined treatment event. The system further applies a plurality of algorithms to detected signals in parallel to arrive at a plurality of estimates of whether a freeze event is occurring and confidences associated with the estimates. The estimates are used to evaluate whether the freeze event is in fact occurring.

### CROSS-REFERENCE TO RELATED APPLICATION

The present application claims the benefit of and priority under 35 U.S.C. §119(e) to U.S. Provisional Patent Application No. 62/153,896, filed Apr. 28, 2015, which is incorporated herein by reference in its entirety.

### INCORPORATION BY REFERENCE OF COMMONLY-OWNED APPLICATIONS AND PATENTS

The following commonly assigned U.S. Patent Applications and U.S. Patents are incorporated herein by reference in their entireties:

U.S. Patent Publication No. 2008/0287839 entitled “METHOD OF ENHANCED REMOVAL OF HEAT FROM SUBCUTANEOUS LIPID-RICH CELLS AND TREATMENT APPARATUS HAVING AN ACTUATOR”;

U.S. Pat. No. 6,032,675 entitled “FREEZING METHOD FOR CONTROLLED REMOVAL OF FATTY TISSUE BY LIPOSUCTION”;

U.S. Patent Publication No. 2007/0255362 entitled “CRYOPROTECTANT FOR USE WITH A TREATMENT DEVICE FOR IMPROVED COOLING OF SUBCUTANEOUS LIPID-RICH CELLS”;

U.S. Pat. No. 7,854,754 entitled “COOLING DEVICE FOR REMOVING HEAT FROM SUBCUTANEOUS LIPID-RICH CELLS”;

U.S. Patent Publication No. 2011/0066216 entitled “COOLING DEVICE FOR REMOVING HEAT FROM SUBCUTANEOUS LIPID-RICH CELLS”;

U.S. Patent Publication No. 2008/0077201 entitled “COOLING DEVICES WITH FLEXIBLE SENSORS”;

U.S. Patent Publication No. 2008/0077211 entitled “COOLING DEVICE HAVING A PLURALITY OF CONTROLLABLE COOLING ELEMENTS TO PROVIDE A PREDETERMINED COOLING PROFILE”;

U.S. Patent Publication No. 2009/0118722, filed Oct. 31, 2007, entitled “METHOD AND APPARATUS FOR COOLING SUBCUTANEOUS LIPID-RICH CELLS OR TISSUE”;

U.S. Patent Publication No. 2009/0018624 entitled “LIMITING USE OF DISPOSABLE SYSTEM PATIENT PROTECTION DEVICES”;

U.S. Patent Publication No. 2009/0018623 entitled “SYSTEM FOR TREATING LIPID-RICH REGIONS”;

U.S. Patent Publication No. 2009/0018625 entitled “MANAGING SYSTEM TEMPERATURE TO REMOVE HEAT FROM LIPID-RICH REGIONS”;

U.S. Patent Publication No. 2009/0018627 entitled “SECURE SYSTEM FOR REMOVING HEAT FROM LIPID-RICH REGIONS”;

U.S. Patent Publication No. 2009/0018626 entitled “USER INTERFACES FOR A SYSTEM THAT REMOVES HEAT FROM LIPID-RICH REGIONS”;

U.S. Pat. No. 6,041,787 entitled “USE OF CRYOPROTECTIVE AGENT COMPOUNDS DURING CRYOSURGERY”;

U.S. Pat. No. 8,285,390 entitled “MONITORING THE COOLING OF SUBCUTANEOUS LIPID-RICH CELLS, SUCH AS THE COOLING OF ADIPOSE TISSUE”;

U.S. Provisional Patent Application Ser. No. 60/941,567 entitled “METHODS, APPARATUSES AND SYSTEMS FOR COOLING THE SKIN AND SUBCUTANEOUS TISSUE”;

U.S. Pat. No. 8,275,442 entitled “TREATMENT PLANNING SYSTEMS AND METHODS FOR BODY CONTOURING APPLICATIONS”;

U.S. patent application Ser. No. 12/275,002 entitled “APPARATUS WITH HYDROPHILIC RESERVOIRS FOR COOLING SUBCUTANEOUS LIPID-RICH CELLS”;

U.S. patent application Ser. No. 12/275,014 entitled “APPARATUS WITH HYDROPHOBIC FILTERS FOR REMOVING HEAT FROM SUBCUTANEOUS LIPID-RICH CELLS”;

U.S. Patent Publication No. 2010/0152824 entitled “SYSTEMS AND METHODS WITH INTERRUPT/RESUME CAPABILITIES FOR COOLING SUBCUTANEOUS LIPID-RICH CELLS”;

U.S. Pat. No. 8,192,474 entitled “TISSUE TREATMENT METHODS”;

U.S. Patent Publication No. 2010/0280582 entitled “DEVICE, SYSTEM AND METHOD FOR REMOVING HEAT FROM SUBCUTANEOUS LIPID-RICH CELLS”;

U.S. Patent Publication No. 2012/0022518 entitled “COMBINED MODALITY TREATMENT SYSTEMS, METHODS AND APPARATUS FOR BODY CONTOURING APPLICATIONS”;

U.S. Publication No. 2011/0238050 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;

U.S. Publication No. 2011/0238051 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;

U.S. Publication No. 2012/0239123 entitled “DEVICES, APPLICATION SYSTEMS AND METHODS WITH LOCALIZED HEAT FLUX ZONES FOR REMOVING HEAT FROM SUBCUTANEOUS LIPID-RICH CELLS”;

U.S. patent application Ser. No. 13/830,413 entitled “MULTI-MODALITY TREATMENT SYSTEMS, METHODS AND APPARATUS FOR ALTERING SUBCUTANEOUS LIPID-RICH TISSUE”; and

U.S. patent application Ser. No. 13/830,027 entitled “TREATMENT SYSTEMS WITH FLUID MIXING SYSTEMS AND FLUID-COOLED APPLICATORS AND METHODS OF USING THE SAME”.

### TECHNICAL FIELD

The present invention relates generally to systems and methods for monitoring a treatment site while cooling/heating tissue. Several embodiments are directed to identifying freeze events and to controlling treatment devices based on the identification of freeze events.

### BACKGROUND

Excess body fat, or adipose tissue, may be present at various locations of a subject's body and may detract from personal appearance. Excess adipose tissue is thought to magnify the unattractive appearance of cellulite, which forms when subcutaneous fat protrudes into the dermis and creates dimples where the skin is attached to underlying structural fibrous strands. Cellulite and excessive amounts of adipose tissue are often considered to be unappealing. Moreover, significant health risks may be associated with higher amounts of excess body fat.

Conventional non-invasive treatments for reducing adipose tissue often include regular exercise, application of topical agents, use of weight-loss drugs, dieting, or a combination of these treatments. One drawback of these non-invasive treatments is that they may not be effective or even possible under certain circumstances. For example, when a person is physically injured or ill, regular exercise may not be an option. Topical agents and orally administered weight-loss drugs are not an option if, as another example, they cause an undesirable reaction (e.g., an allergic or negative reaction).

A variety of non-invasive methods have been used to treat individuals having excess body fat. Non-invasive methods for reducing adipose tissue can include applying, e.g., radiofrequency (“RF”) and/or light energy, such as described in U.S. Patent Publication No. 2006/0036300 and U.S. Pat. No. 5,143,063, or applying, e.g., high intensity focused ultrasound (HIFU) radiation, such as described in U.S. Pat. Nos. 7,258,674 and 7,347,855. Non-invasive cooling of subcutaneous tissue can also reduce adipose tissue. Conventional cooling systems often have thermoelectric devices that can be placed against a patient's skin. The thermoelectric devices can conductively remove heat from the subject's skin to cool and reduce underlying subcutaneous tissue. Unfortunately, conventional cooling systems cannot accurately identify various events during treatment. Such events can include, for example, freezing of skin, movement of thermoelectric devices, or other unwanted events that may adversely affect treatment. Accordingly, conventional cooling systems often do not comfortably and consistently treat patients.

### SUMMARY OF THE INVENTION

At least some embodiments of the invention are directed to systems and methods of monitoring cryotherapy. Systems disclosed herein can utilize one or more filters to evaluate whether an event has occurred or will occur and can perform one or more actions based on the determination. In some embodiments, systems can shut off a treatment device, adjust heating/cooling rates, or otherwise alter operation of the treatment device based on the determination. For example, if the system determines that a partial freeze event has occurred, the system can reduce the rate of heat removal or turn off the treatment device to, for example, allow frozen tissue to thaw, inhibit addition freezing, and/or otherwise manage thermal effects. Other actions can be taken for other events.

The filters can be adaptive filters for enhancing detection accuracy by processing output from sensors. Additionally or alternatively, sensor output can be processed using one or more algorithms to estimate whether an event will occur or has occurred and, in some embodiments, to produce multiple confidences (e.g., 3, 4, or 5 confidences) associated with such estimates. The event can be a partial freeze event, complete freeze event, false detection event, or other event that may affect treatment. The system can be programmed to detect any number of different events, and the estimates can be used to evaluate whether events of interest have actually occurred.

In some embodiments, a non-invasive treatment device for removing heat from a subject's tissue comprises a treatment device, a first sensor, a signal processor, and a controlling device. The treatment device can be configured to contact an area of the subject's skin and remove heat from tissue located below the contacted area of skin. For example, heat can be removed from subcutaneous adipose tissue below the contacted area. The first sensor can be configured to measure one or more characteristics of the treatment device, subcutaneous tissue, and/or the skin and can be configured to output first signals based upon the measured characteristic(s). The signal processor can be programmed to estimate characteristics based on the first signals. Such estimated characteristics can be noise characteristics generated using an adaptive filter that provides at least one filtered first signal. The signal processor can use the filtered first signal to determine, among other things, whether at least a partial freeze event will occur, whether at least a partial freeze event has occurred, or other event information. The controlling device can then modify operation of the treatment device based on the determination by the signal processor.

The signal processor can be programmed to dynamically change its transfer function to remove varying amounts of noise from the first signal based on, for example, estimated noise characteristic(s). Different algorithms can be used to change the transfer function to achieve the desired noise removal. In some embodiments, the signal processor includes, without limitation, one or more adaptive low pass filters, Kalman filters, and/or adaptive noise cancellers. By way of example, an adaptive low pass filter can average first signals from the first sensor, and the averaging can be dependent, at least in part, on estimated noise characteristics. As the estimated noise characteristics change, the averaging algorithm applied to the first signals can be correspondingly changed. In embodiments in which the signal processor includes the Kalman filter, the Kalman filter can generate a plurality of measurements derived from the first signal. The Kalman filter can compare selected measurements with at least one expected measurement. Variable weights can be assigned to each selected measurement based on comparison steps so as to generate weighted measurements. Variable weights can be assigned in response to identified similarities between each selected measurement and a corresponding previous measurement and based on, for example, expected measurements. The weighted measurements can be averaged to obtain a filtered measurement for use in estimating the likelihood of a freeze event (e.g., a partial freeze event, a complete freeze event, etc.) or other event. The Kalman filter can adjust averaging and/or assigning processes to increase accuracy.

In other embodiments, a non-invasive treatment system for transdermally removing heat from tissue beneath the subject's skin is comprised of a treatment device, a first sensor, and a signal processor. The treatment device can be configured to perform a wide range of different cryotherapy procedures. The first sensor can detect a characteristic of the treatment device, subcutaneous tissue, and/or skin. For example, the detected characteristic can be a temperature indicative of the temperature of cooled skin, temperature of tissue below cooled skin, and/or temperature of a cooling surface of the treatment device. The first sensor can be configured to output one or more first signals that can be used to determine information about an event. Such information can include, for example, whether the event has occurred or will occur or other event information. In one embodiment, the signal processor is programmed to estimate at least one noise characteristic of one or more first signals to determine whether a partial freeze event has occurred. The determination can be based, at least in part, on estimated noise characteristics such that a freeze event is determined to not have occurred when the estimated noise characteristics exceed a predetermined noise characteristic value. The predetermined noise characteristic value can be selected based on, for example, characteristics of the first signals, user settings, patient history, or the like.

Estimated noise characteristics can be derived, at least in part, by comparing the first signals to at least one reference signal value. The reference signal values can be signals derived from stored data, which can include, for example, empirical data, signal templates, patient data, and/or other information. The signal processor can also be programmed to determine that no freeze event has occurred when the estimated noise characteristic is too large. When the first signals experience excessive noise, the system can thus avoid erroneously detecting freeze events that have not occurred.

In yet other embodiments, a non-invasive treatment system for transdermally removing heat from tissue of a subject comprises a treatment device, a sensor, and a controlling device. The sensor can measure a characteristic of the treatment device, subcutaneous tissue, and/or the subject's skin. The sensor can be configured to output one or more signals that are received by the controlling device. The controlling device can control the treatment device based upon the signals. For example, the controlling device can analyze the signals to determine whether an event, such as a partial freeze event, has occurred. In one embodiment, the controlling device includes a signal processor programmed to determine a first value using a first filtering algorithm applied to one or more of the output signals, determine a second value using a second filtering algorithm applied to one or more of the output signals, and/or determine whether an event has occurred or is occurring based on the first and second values. In embodiments for detecting freeze events, the first value can be a first likelihood of a freeze event occurring and the second value can be a second likelihood of the freeze event occurring. The first and second likelihoods can be compared or otherwise analyzed to determine, for example, whether a freeze event has occurred, a freeze event is occurring, or other freeze event information.

In some embodiments, a method for removing heat from tissue beneath skin and detecting a freeze event in the presence of motion is provided. The method can include removing heat from tissue located below a skin surface using a treatment device. One or more characteristics of at least one of the treatment device, the tissue, and/or the skin can be measured using a sensor. A signal can be adaptively filtered to eliminate motion artifact and read through motion induced signal noise. The method can include determining whether a freeze event is or has occurred based on analysis of the filtered signal.

In some further embodiments, a non-invasive treatment system for treating a target site comprises a treatment device configured to cool targeted tissue, deliver energy to targeted tissue, and/or otherwise affect targeted tissue. The treatment system can include, without limitation, one or more sensors, signal processors, and controlling devices. The sensors can detect a characteristic of the treatment device, tissue (e.g., subcutaneous tissue, skin, etc.), and/or components or features of the treatment system. The signal processor can be programmed to estimate one or more characteristics of signals from the sensor. Based on the estimated characteristic(s), the signal processor can adaptively process (e.g., filter, modify, etc.) the signals to provide at least one processed signal. For example, estimated characteristic(s) can include, without limitation, one or more noise characteristics, error characteristics, or other characteristics. The signal processor can use the processed signal(s) to, for example, determine whether one or more events have occurred and/or otherwise monitor treatment. In some embodiments, the signal processor can filter the signals to create filtered signals to detect one of more events and read through motion and other signal noise. The controlling device can control the treatment device based, at least in part, upon occurrence of a certain event. In some embodiments, the treatment device removes heat from subcutaneous tissue, and the signal processor can analyze signals to determine, for example, whether one or more events will occur or have occurred due to heat removal by the treatment device. These events can include, without limitation, freeze events (e.g., partial freeze events, complete freeze events, etc.), lift off events, and/or other events associated with cooling/heating tissue.

### BRIEF DESCRIPTION OF THE DRAWINGS

In the drawings, identical reference numbers identify similar elements or acts.

FIG. 1 is a partially schematic, isometric view of a treatment system for non-invasively affecting target regions of a subject in accordance with an embodiment of the invention.

FIG. 2 is a cross-sectional view of a connector of the treatment system taken along line 2-2 of FIG. 1.

FIG. 3 is a side view of a treatment device in accordance with an embodiment of the invention.

FIG. 4 is a time versus skin temperature graph for a tissue cooling procedure showing a freeze event.

FIG. 5 is another time versus skin temperature graph for a tissue cooling procedure showing another freeze event.

FIG. 6 is a time versus temperature graph with actual and measured skin temperatures during a cryotherapy procedure.

FIG. 7 shows an adaptive noise canceller in accordance with various embodiments of the present invention.

FIG. 8 illustrates a parallel processing system in accordance with various embodiments of the present invention.

FIG. 9 is a schematic block diagram illustrating subcomponents of a treatment system in accordance with embodiments of the present invention.

### DETAILED DESCRIPTION

### A. Overview

The present invention describes treatment systems and methods for predicting, detecting, and/or monitoring events associated with cooling/heating tissue. Several embodiments are directed to methods for detecting freeze events during a cryotherapy procedure. For example, tissue can be monitored to identify freeze events in the skin during cooling of subcutaneous adipose tissue. The treatment system can modify treatment to stop the identified events, inhibit the occurrence of future undesired events, and/or alter (e.g., minimize, limit, or maximize) the effects of the events. Several of the details set forth below are provided to describe the following examples and methods in a manner sufficient to enable a person skilled in the relevant art to practice, make, and use them. Several of the details and advantages described below, however, may not be necessary to practice certain examples and methods of the invention. Additionally, the invention may include other examples and methods that are within the scope of the invention but are not described in detail.

Some of the embodiments disclosed herein can be for cosmetically beneficial alterations of target regions. Some cosmetic procedures may be for the sole purpose of altering a target region to conform to a cosmetically desirable look, feel, size, shape and/or other desirable cosmetic characteristic or feature. Accordingly, at least some embodiments of the cosmetic procedures can be performed and monitored without providing an appreciable therapeutic effect (e.g., no therapeutic effect). For example, some cosmetic procedures may not include restoration of health, physical integrity, or the physical well-being of a subject. The cosmetic methods can target subcutaneous regions to change a subject's appearance and can include, for example, procedures performed on subject's hips, legs, waist, stomach, submental region, face, neck, ankle region, or the like. In other embodiments, however, cosmetically desirable treatments may have therapeutic outcomes (whether intended or not), such as psychological benefits, alteration of body hormones levels (by the reduction of adipose tissue), etc. The treatment system can monitor procedures to minimize, limit, and/or substantially prevent unwanted affects or events. For example, cooling of tissue can be monitored to minimize or avoid freezing.

Reference throughout this specification to “one example,” “an example,” “one embodiment,” or “an embodiment” means that a particular feature, structure, or characteristic described in connection with the example is included in at least one example of the present invention. Thus, the occurrences of the phrases “in one example,” “in an example,” “one embodiment,” or “an embodiment” in various places throughout this specification are not necessarily all referring to the same example. Furthermore, the particular features, structures, routines, stages, or characteristics may be combined in any suitable manner in one or more examples of the invention. The headings provided herein are for convenience only and are not intended to limit or interpret the scope or meaning of the invention.

### B. Cryotherapy

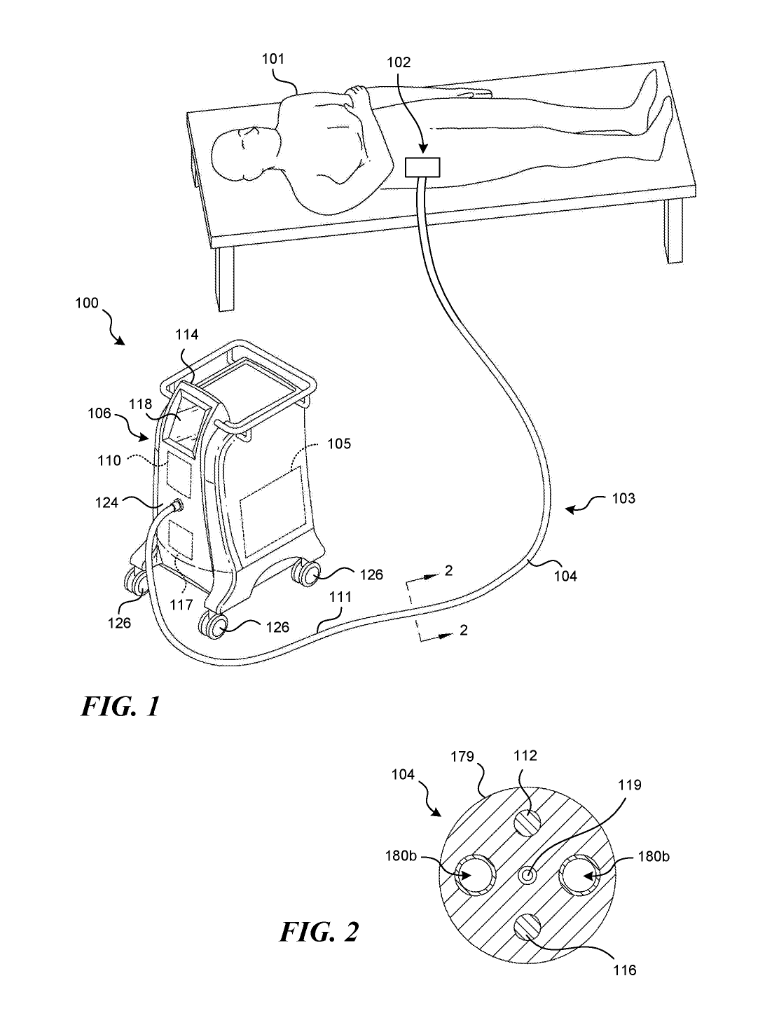

FIG. 1 and the following discussion provide a brief, general description of a treatment system 100 in accordance with some embodiments of the invention. The treatment system 100 can be a temperature-controlled system for exchanging heat with a subject 101 and can include a non-invasive applicator or treatment device 102 (“treatment device 102”) configured to selectively cool/heat tissue to reduce and/or eliminate targeted tissue to achieve a desired overall appearance. The treatment system 100 can monitor treatment and, in some embodiments, modifies operation of the treatment device 102 based on detected events. In some non-invasive cryotherapy procedures, the treatment site can be monitored to avoid or limit freeze events in the skin while cooling targeted subcutaneous tissue.

Without being bound by theory, the selective effect of cooling is believed to result in, for example, membrane disruption, cell shrinkage, disabling, disrupting, damaging, destroying, removing, killing, and/or other methods of lipid-rich cell alteration. Such alteration is believed to stem from one or more mechanisms acting alone or in combination. It is thought that such mechanism(s) trigger an apoptotic cascade, which is believed to be the dominant form of lipid-rich cell death by non-invasive cooling. In any of these embodiments, the effect of tissue cooling can be the selective reduction of lipid-rich cells by a desired mechanism of action, such as apoptosis, lipolysis, or the like. In some procedures, the treatment device 102 can cool the tissue of the subject 101 (e.g., a human or an animal) to a temperature in a range of from about −40° C. to about 20° C. In other embodiments, the cooling temperatures can be from about −20° C. to about 10° C., from about −18° C. to about 5° C., from about −15° C. to about 5° C., or from about −15° C. to about 0° C. In further embodiments, the cooling temperatures can be equal to or less than −5° C., −10° C., −15° C., or in yet another embodiment, from about −15° C. to about −40° C. Other cooling temperatures and temperature ranges can be used.

Apoptosis, also referred to as “programmed cell death”, is a genetically-induced death mechanism by which cells self-destruct without incurring damage to surrounding tissues. An ordered series of biochemical events induce cells to morphologically change. These changes include cellular blebbing, loss of cell membrane asymmetry and attachment, cell shrinkage, chromatin condensation and chromosomal DNA fragmentation. Injury via an external stimulus, such as cold exposure, is one mechanism that can induce cellular apoptosis in cells. Nagle, W. A., Soloff, B. L., Moss, A. J. Jr., Henle, K. J. “Cultured Chinese Hamster Cells Undergo Apoptosis After Exposure to Cold but Nonfreezing Temperatures” Cryobiology 27, 439-451 (1990).

One aspect of apoptosis, in contrast to cellular necrosis (a traumatic form of cell death causing local inflammation), is that apoptotic cells express and display phagocytic markers on the surface of the cell membrane, thus marking the cells for phagocytosis by macrophages. As a result, phagocytes can engulf and remove the dying cells (e.g., the lipid-rich cells) without eliciting an immune response. Temperatures that elicit these apoptotic events in lipid-rich cells may contribute to long-lasting and/or permanent reduction and reshaping of subcutaneous adipose tissue.

One mechanism of apoptotic lipid-rich cell death by cooling is believed to involve localized crystallization of lipids within the adipocytes at temperatures that do not induce crystallization in non-lipid-rich cells. The crystallized lipids selectively may injure these cells, inducing apoptosis (and may also induce necrotic death if the crystallized lipids damage or rupture the bi-lipid membrane of the adipocyte). Another mechanism of injury involves the lipid phase transition of those lipids within the cell's bi-lipid membrane, which results in membrane disruption or dysfunction, thereby inducing apoptosis. This mechanism is well-documented for many cell types and may be active when adipocytes, or lipid-rich cells, are cooled. Mazur, P., “Cryobiology: the Freezing of Biological Systems” Science, 68: 939-949 (1970); Quinn, P. J., “A Lipid Phase Separation Model of Low Temperature Damage to Biological Membranes” Cryobiology, 22: 128-147 (1985); Rubinsky, B., “Principles of Low Temperature Preservation” Heart Failure Reviews, 8, 277-284 (2003). Other possible mechanisms of adipocyte damage, described in U.S. Pat. No. 8,192,474, relate to ischemia/reperfusion injury that may occur under certain conditions when such cells are cooled as described herein. For instance, during treatment by cooling as described herein, the targeted adipose tissue may experience a restriction in blood supply and thus be starved of oxygen due to isolation as a result of applied pressure, cooling which may affect vasoconstriction in the cooled tissue, or the like. In addition to the ischemic damage caused by oxygen starvation and the buildup of metabolic waste products in the tissue during the period of restricted blood flow, restoration of blood flow after cooling treatment may additionally produce reperfusion injury to the adipocytes due to inflammation and oxidative damage that is known to occur when oxygenated blood is restored to tissue that has undergone a period of ischemia. This type of injury may be accelerated by exposing the adipocytes to an energy source (via, e.g., thermal, electrical, chemical, mechanical, acoustic, or other means) or otherwise increasing the blood flow rate in connection with or after cooling treatment as described herein. Increasing vasoconstriction in such adipose tissue by, e.g., various mechanical means (e.g., application of pressure or massage), chemical means or certain cooling conditions, as well as the local introduction of oxygen radical-forming compounds to stimulate inflammation and/or leukocyte activity in adipose tissue may also contribute to accelerating injury to such cells. Other yet-to-be understood mechanisms of injury may exist.

In addition to the apoptotic mechanisms involved in lipid-rich cell death, local cold exposure is also believed to induce lipolysis (i.e., fat metabolism) of lipid-rich cells and has been shown to enhance existing lipolysis which serves to further increase the reduction in subcutaneous lipid-rich cells. Vallerand, A. L., Zamecnik. J., Jones, P. J. H., Jacobs, I. “Cold Stress Increases Lipolysis, FFA Ra and TG/FFA Cycling in Humans” Aviation, Space and Environmental Medicine 70, 42-50 (1999).

One expected advantage of the foregoing techniques is that the subcutaneous lipid-rich cells in the target region can be reduced generally without collateral damage to non-lipid-rich cells in the same region. In general, lipid-rich cells can be affected at low temperatures that do not affect non-lipid-rich cells. As a result, lipid-rich cells, such as those associated with highly localized adiposity (e.g., submental adiposity, submandibular adiposity, facial adiposity, etc.), can be affected while non-lipid-rich cells (e.g., myocytes) in the same generally region are not damaged. The unaffected non-lipid-rich cells can be located underneath lipid-rich cells (e.g., cells deeper than a subcutaneous layer of fat), in the dermis, in the epidermis, and/or at other locations. The treatment systems disclosed herein can monitor treatment to avoid damaging the non-lipid-rich cells.

In some procedures, the treatment system 100 can remove heat from underlying tissue through the upper layers of tissue and create a thermal gradient with the coldest temperatures near the cooling surface of the treatment device 102 (i.e., the temperature of the upper layer(s) of the skin can be lower than that of the targeted underlying target cells). It may be challenging to reduce the temperature of the targeted cells low enough to be destructive to these target cells (e.g., induce apoptosis, cell death, etc.) while also maintaining the temperature of the upper and surface skin cells high enough so as to be protective (e.g., non-destructive). The temperature difference between these two thresholds can be small (e.g., about 5° C. to about 10° C., less than 10° C., less than 15° C., etc.). Adaptive tissue monitoring can be used to accurately monitor the skin to avoid freeze damage. Additionally or alternatively, protection of the overlying cells (e.g., typically water-rich dermal and epidermal skin cells) from freeze damage during dermatological and related aesthetic procedures that involve sustained exposure to cold temperatures may include improving the freeze tolerance and/or freeze avoidance of these skin cells by using, for example, cryoprotectants for inhibiting or preventing such freeze damage.

The treatment devices can be used to perform a wide range of different cryotherapy procedures. Although many cryotherapy procedures disclosed herein involve preventing partial or complete freezing of tissue, other cryotherapy procedures can be designed to produce freeze events (e.g., at least partially freezing tissue or totally freezing tissue) to elicit a desired response. For example, freeze events can be identified to control the durations of the freeze events, amount of freezing (e.g., extent of freezing in a region of tissue), or the like. Freeze events can involve forming crystals that alter targeted cells to cause skin tightening, skin thickening, fibrosis, or otherwise alter tissue without destroying a significant amount of cells in the skin. To avoid destroying skin cells, the surface of the patient's skin can be cooled for a duration short enough to avoid, for example, excessive ice formation, permanent thermal damage, or significant hyperpigmentation or hypopigmentation (including long-lasting or permanent hyperpigmentation or hypopigmentation). Adaptive tissue monitoring can be used to accurately identify and monitor freeze events. Destruction of skin cells (or excessive damage) can be avoided by applying heat to the surface of the patient's skin to heat the skin cells above their freezing temperature. The patient's skin can be warmed to avoid, for example, excessive ice formation, permanent thermal damage, or significant hyperpigmentation or hypopigmentation of non-targeted tissue, such as epidermal tissue. Such warming processes can be monitored using adaptive techniques to avoid excessive heating that would cause, for example, necrosis.

In some tissue-freezing procedures, the treatment system 100 can controllably cool tissue while monitoring for freeze events. After detecting a freeze event, the treatment system 100 can periodically or continuously remove heat from the target tissue to keep a volume of target tissue frozen or partially frozen for a suitable length of time to elicit a desired response. In some embodiments, controlled freezing can cause tightening of the skin, thickening of the skin, and/or a cold shock response at the cellular level in the skin. In one tissue-freezing procedure, the treatment device 102 can produce a partial or total freeze event in the patient's skin for a relatively short time limit to avoid cooling the adjacent subcutaneous tissue to a low enough temperature to cause subcutaneous cell death or undue injury. Some partial freeze events can include freezing mostly extracellular material without freezing a substantial amount of intercellular material. In other procedures, partial freeze events can include freezing mostly intercellular material without freezing a substantial amount of extracellular material.

### C. Treatment Systems

FIG. 1 shows the treatment system 100 that can include the treatment device 102, a connector 104, and a control module 106 for controlling operation of the treatment device 102. The treatment device 102 can be a vacuum or non-vacuum applicator for performing cryotherapy or other procedures and can include, without limitation, one or more sensors used to, for example, monitor treatment. The connector 104 can be an umbilical cord that provides communication between the treatment device 102 and the control module 106. For example, the connector 104 can provide communication between sensors of the treatment device 102 and the control module 106.

The treatment device 102 can cool subcutaneous tissue of the subject 101 to reduce or eliminate subcutaneous adipose tissue while non-targeted tissue can be generally unaffected. Sensors of the treatment device 102 can be used to, for example, detect and/or monitor events before, during, and/or after removal of heat from targeted tissue. Treatment devices may be designed to treat particular sites along the patient's body, such as the chin, cheeks, arms, pectoral areas, thighs, calves, buttocks, abdomen, “love handles”, back, submental tissue, and so forth. For example, treatment devices (e.g., vacuum applicators) may be applied at the stomach or back region, and other treatment devices (e.g., belt applicators) can be applied around the thigh region. Exemplary treatment devices and their configurations and components usable with the treatment system 100 are described in, e.g., commonly assigned U.S. Patent Publication Nos. 2007/0198071, 2008/0077201, and 2008/0077211 and in U.S. patent application Ser. No. 11/750,953. In further embodiments, the treatment system 100 may also include a patient protection device (not shown) incorporated into or configured for use with the treatment device 102 to prevent the treatment device from directly contacting a patient's skin and thereby reducing the likelihood of cross-contamination between patients and/or minimizing cleaning requirements for the treatment device. Patient protection devices may also include or incorporate various storage, computing, and communications devices, such as a radio frequency identification (RFID) component, allowing for example, use to be monitored and/or metered. Exemplary patient protection devices are described in commonly assigned U.S. Patent Publication No. 2008/0077201.

FIG. 1 shows the connector 104 extending from the control module 106 to the treatment device 102. The connector 104 can provide, for example, suction for drawing tissue into or against the treatment device 102, energy (e.g., electrical energy) for powering electronic components, and/or fluid (e.g., coolant) for tissue cooling. The connector 104 can include one or more communication components, electrical lines, fluid lines, lumens, and other components. An embodiment of the connector 104 is discussed in connection with FIG. 2, which is a cross-sectional view of the connector 104 taken along line 2-2 of FIG. 1. The connector 104 of FIG. 2 includes a main body 179, a supply fluid line or lumen 180a (“supply fluid line 180a”), and a return fluid line or lumen 180b (“return fluid line 180b”). The main body 179 may be configured (via one or more adjustable joints) to “set” in place for the treatment of the subject. The supply and return fluid lines 180a, 180b can be conduits comprising, in whole or in part, polyethylene, polyvinyl chloride, polyurethane, and/or other materials that can accommodate circulating coolant, such as water, glycol, synthetic heat transfer fluid, oil, a refrigerant, and/or any other suitable heat conducting fluid. In one embodiment, each fluid line 180a, 180b can be a flexible hose surrounded by the main body 179. The connector 104 can also include one or more electrical lines 112 for providing power to the treatment device 102 and one or more communication components 116 for providing communication between the control module 106 (FIG. 1) and components of the treatment device 102 (FIG. 1). The communication component 116 can be, for example, one or more lines, wires, etc. To provide suction, the connector 104 can include one or more vacuum lines 119. In various embodiments, the connector 104 can include a bundle of fluid conduits, a bundle of power lines, wired connections, vacuum lines, and other bundled and/or unbundled components selected to provide ergonomic comfort, minimize unwanted motion (and thus potential inefficient removal of heat from the subject), and/or to provide an aesthetic appearance to the treatment system 100.

Referring again to FIG. 1, the control module 106 can control treatment by, for example, turning off the cooling capability of the treatment device 102, reducing but not turning off the cooling capability of the treatment device 102, adjusting treatment times, and/or alerting a clinician. The control module 106 can include a fluid system 105 (illustrated in phantom line), a power supply 110 (illustrated in phantom line), and a controller or controlling device 114 (“controller 114”) carried by a housing 124 with wheels 126. The fluid system 105 can include a fluid chamber and a refrigeration unit, a cooling tower, a thermoelectric chiller, heaters, or any other device capable of controlling the temperature of coolant in the fluid chamber. The coolant can be continuously or intermittently delivered to the treatment device 102 via the supply fluid line 180a (FIG. 2) and can circulate through the treatment device 102 to absorb heat. The coolant, which has absorbed heat, can flow from the treatment device 102 back to the control module 106 via the return fluid line 180b (FIG. 2). For warming periods, the control module 106 can heat the coolant such that warm coolant is circulated through the treatment device 102. Alternatively, a municipal water supply (e.g., tap water) can be used in place of or in conjunction with the control module 106.

In vacuum-assisted embodiments, a pressurization device 117 can provide suction to the treatment device 102 via the vacuum line 119 (FIG. 2) and can include one or more pumps, vacuum sources, or the like. Air pressure can be controlled by a regulator located between the pressurization device 117 and the treatment device 102. If the vacuum level is too low, tissue may not be drawn adequately (or at all) against or into the treatment device 102. If the vacuum level is too high, undesirable discomfort to the patient 101 and/or tissue damage could occur. The control module 106 can control the vacuum level to draw tissue against or into the treatment device 102 while maintaining a desired level of comfort.

The power supply 110 can provide a direct current voltage for powering electrical elements of the treatment device 102 via the line 112 (FIG. 2). An operator can use an input/output device 118 of the controller 114 to control operation of the treatment system 100, and the input/output device 118 can display the state of operation and progress of a treatment protocol. In some embodiments, the controller 114 can exchange data with the treatment device 102 via wired, wireless, or optical communication links and can adjust treatment based on, without limitation, one or more treatment profiles, patient data, and/or patient-specific treatment plans, such as those described, for example, in commonly assigned U.S. Pat. No. 8,275,442. Each treatment protocol (e.g., treatment template, profile, and plan) can include one or more segments, and each segment can include signal processing routines (e.g., routines to minimize or limit signal noise), target temperature profiles, vacuum levels, and/or specified durations (e.g., 1 minute, 5 minutes, 10 minutes, 20 minutes, 30 minutes, 1 hour, 2 hours, etc.). If the treatment system 100 includes multiple treatment devices, a treatment profile can include specific profiles for each treatment device to concurrently or sequentially treat multiple treatment sites.

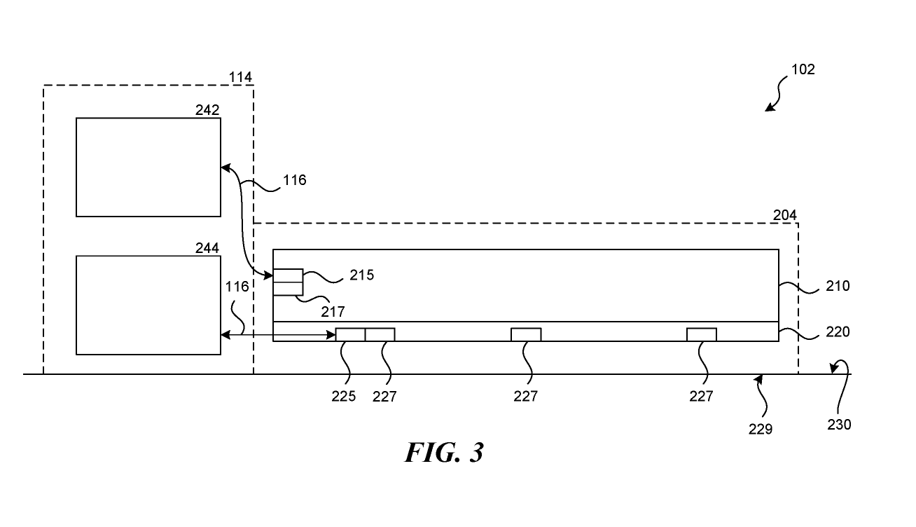

FIG. 3 is a schematic view illustrating a treatment device 102 in accordance with one embodiment. The treatment device 102 may include a cooling unit, such as a cooling plate 210, and an interface layer 220. The cooling plate 210 can include cooling elements (e.g., Peltier devices), cooling channels/passages, or other thermal elements and can contain one or more communication components 215 and at least one sensor 217. The communication components 215 can communicate with, for example, a component 242 of the controller 114. The interface layer 220 may be a plate, a film, a covering, or other suitable material or component and may serve as a patient protection device, and the interface layer 220 may also include one or more communication components 225 and one or more sensors 227. In some embodiments, the communication components 215, 225, and/or both may receive and transmit information, such as one or more characteristics of the treatment device 102, tissue (e.g., targeted tissue, subcutaneous tissue, skin, etc.), or other components. For example, the communication components 215, 225 can be connected to, for example, communication line(s) (e.g., communication line 116 of FIG. 2). The communication component 225 of FIG. 3 can communicate with a component 244 of the controller 114.

The treatment device 102 may include a separate sleeve and/or liner that is used to contact the patient's skin. Further details regarding sleeves, liners, and patient protection devices may be found in U.S. Patent Publication No. 2008/0077201. In some cases, the treatment device 102 may include a device having a belt that assists in forming a contact between the treatment device (such as via an interface layer) and the patient's skin. For example, the treatment device 102 may include retention devices (e.g., belts, straps, etc.). To assist in therapy, the treatment device 102 may provide mechanical energy to a treatment region. Imparting mechanical vibratory energy to the patient's tissue by repeatedly applying and releasing a vacuum to the subject's tissue, for instance, creates a massage action during treatment. Further details regarding a vacuum type device may be found in U.S. patent application Ser. No. 11/750,953.

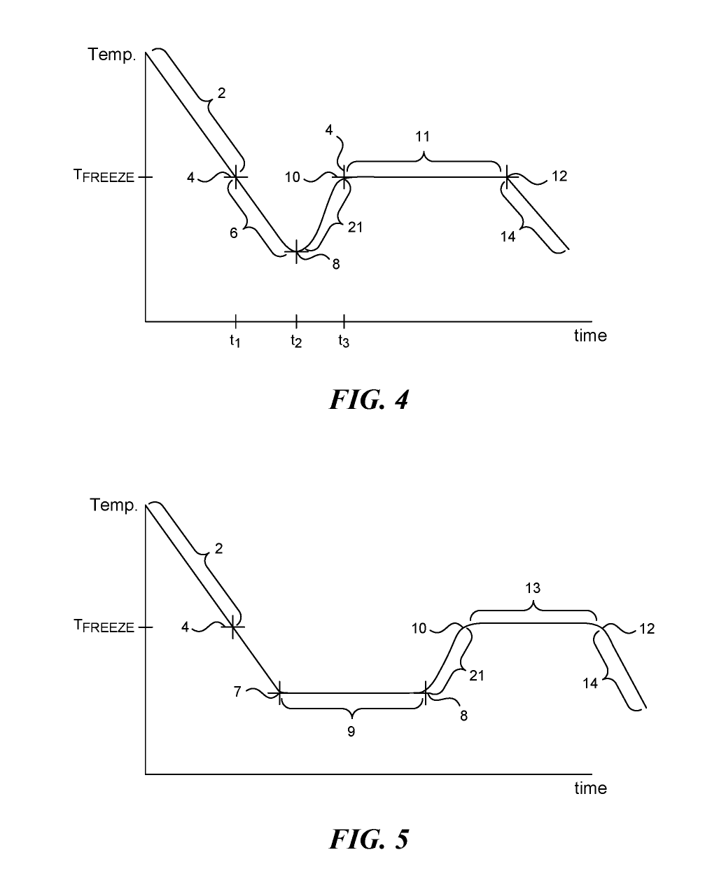

The sensors 217, 227 can be temperature sensors (e.g., thermistors), optical sensors, impedance sensors, motion sensors, accelerometers, vibration sensors, or other types of detectors that can be attached to, embedded in, or otherwise coupled to the interface layer 220, plate 210, and/or other component of the treatment device 102. The sensors 217, 227 can be used to monitor the treatment site and, in some embodiments, to detect events discussed in connection with FIGS. 4 to 6 to minimize or avoid under treatment, over treatment, prematurely terminating treatment, and/or unwanted events. The detected events can be supercooling events, freeze events, or other events associated with therapy.

FIG. 4 is a time versus temperature graph during a cooling treatment in which the skin is continually cooled and a freeze event 8 begins to occur, either by design or inadvertently. As the skin is initially cooled, its temperature falls in zone 2 from an initial value at time 0 to a value equal to its freezing temperature 4 (e.g., about −0.5° C. to about −1.8° C.) at time t₁. The initial value at time 0 can be a temperature between the subject's body temperature and room temperature or a pre-warming or pre-cooling temperature. For example, the initial temperature of the subject's skin can be at, e.g., about 32° C. to about 34° C.

As cooling continues after reaching the skin's freezing temperature at t₁, oftentimes the skin does not immediately freeze and instead enters a supercooled zone 6 where the skin temperature declines below skin's freezing temperature but freezing of the skin does not occur. If cooling continues, the skin can be cooled from freezing temperature 4 at time t₁ to point 8 (time t₂) at which point the freeze event begins. Once freezing or crystallization begins and continues as shown by zone 21, the skin can rapidly partially freeze until the latent heat of fusion released raises a bulk temperature of the skin to a value 10 at time t₃. The value 10 is generally approximately equal to the skin freezing temperature 4, T_{freeze}. Hence, the bulk temperature of the skin at point 10 is such that the supercooled state no longer exists. If cooling continues indefinitely beyond this point 10, then the amount of partial freezing existent in the skin can gradually increase while the temperature of the skin can remain relatively constant at T_{freeze}. If cooling is continued after point 10, most of the previously unfrozen skin in thermal contact with the treatment device gradually becomes frozen while in zone 11 until point 12 is reached whereby the skin is completely frozen. Thereafter, if cooling continues, the temperature of the totally frozen skin as represented in zone 14 can decline. In practice, complete freezing of skin (e.g., 100 percent of the skin under the treatment device) is often not allowed to occur or desired with the use of a treatment system that employs transdermal surface cooling of bulk skin tissue. This is because substantial skin tissue damage could result and the treatment would be unduly painful. One aim of at least some disclosed treatment systems is to either prevent any freezing or allow only partial freezing to occur and then strictly limit and control an amount of partial freezing which is allowed to occur to levels deemed acceptably safe and/or comfortable. For example, treatment systems disclosed herein can detect freeze events shown in FIG. 4 and modify its operation to manage freezing, if any.

FIG. 5 is a time versus temperature graph similar to FIG. 4, except in this instance the skin is not as deeply cooled, as in FIG. 4, from its initial state so as to attempt to not cause any freezing whatsoever during the treatment. Similar to FIG. 4, zone 2 of FIG. 5 shows the period during which skin is cooled from a starting temperature at time 0 to the skin freezing temperature 4 whereat supercooling begins. However, in this example, at supercooled point 7, an amount of cooling is controlled (e.g., limited) so as to attempt to maintain a relatively constant supercooled temperature in zone 9 throughout the entire treatment without freezing any tissue. So the aim in this embodiment is to keep a supercooled state of the skin in zone 9 during a totality of the treatment to achieve beneficial tissue effects (which are enhanced by maintaining low supercooled temperatures), without having the skin actually freeze, which can cause adverse effects. However, if freezing begins, either intentionally or inadvertently at some point 8, its signature may be evident by the rapid rise in skin temperature in zone 21 caused by the release of heat due to the latent heat of fusion associated with crystallization. Other detectable markers for the freeze event could also be detected, such as, for example, a change in the optical or electrical properties of the skin. The release of heat can cause the temperature of the skin to rise to its freezing temperature at point 10. Again, at point 10, if cooling continues, the temperature of the skin can remain relatively constant at T_{freeze} in zone 13 until point 12 where all the skin (i.e., all the skin in thermal contact with the treatment device) can be substantially frozen if an amount of cooling being delivered to the patient is not altered. In practice with non-invasive transdermal cooling devices which cool bulk skin tissue, total freezing and sometimes even any partial freezing is often not desired, and designs are often implemented to prevent such events.

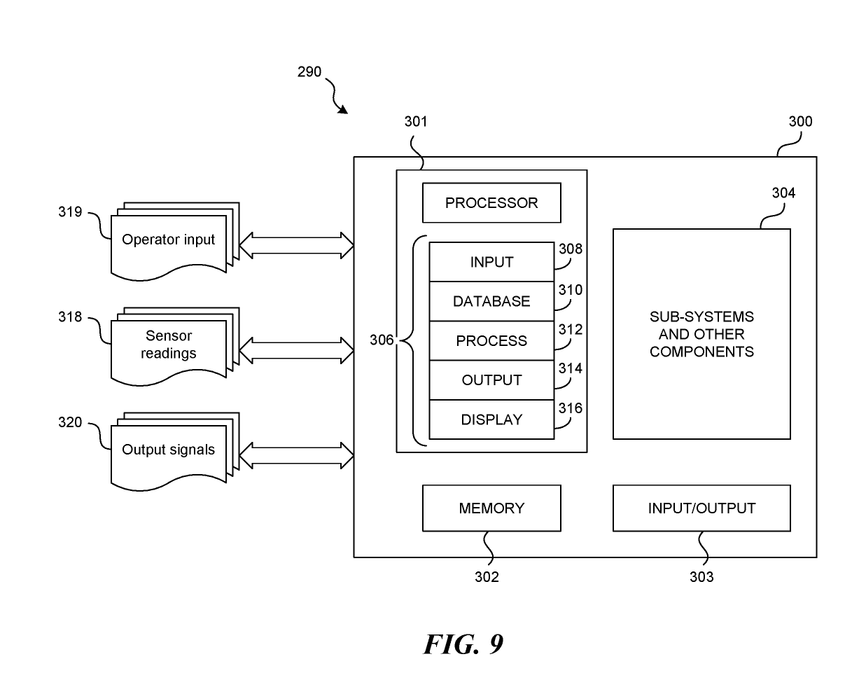

The treatment systems disclosed herein can detect the freeze events 21 discussed in connection with FIGS. 4 and 5 because it is often desirable to reliably and accurately detect if a partial or total freeze event begins or is occurring. If a freeze event is detected, it may be desirable for a controller (e.g., controller 114 of FIG. 1, controller 290 of FIG. 9, etc.) to either shut off the treatment device, end the procedure, alert an operator, and/or adjust (or limit) an amount and duration of further cooling. For example, treatment systems disclosed herein can monitor tissue cooling to detect supercooling, partial freezing (e.g., partial freezing which begins at point 10 in FIGS. 4 and 5), complete freezing (e.g., freezing at zones 14 in FIGS. 4 and 5), and so forth.

Temperature sensors and other means can be used to measure skin temperatures, component temperatures, or other characteristics of the treatment device or skin. Many factors can affect the accuracy of all these measurements and may result in, for example, false measurements, false event detections, etc. By way of example, if a patient moves during treatment or if an applicator attached to the patient moves for any reason, the applicator may lift off the patient. If temperature sensors (e.g., sensors 217, 227 of FIG. 3) are attached to or are part of the applicator, this lift off can generate false skin temperature readings. Generally, upon lift off, a temperature sensor 217, 227 on or in the applicator can begin to detect (e.g., measure, record, etc.) a drop in temperature because air is a relatively good insulator and the sensor temperature can converge with the applicator temperature, which is lower than a temperature of the skin being treated. Upon re-contact with the skin, the temperature of the sensor can then rise to approximate the higher temperature of the skin being treated, since this skin is warmer than the cooling-surface of the applicator (e.g., cooling surface 229 of FIG. 3). This detected temperature rise can be similar to zone 21 in FIGS. 4, 5 and can be misinterpreted as a freeze event when in fact no freeze event has occurred. Another erroneous freeze event detection can occur when a pressure of the applicator against the skin changes. For example, a relatively large normal pressure can approximate more normal operation, and a relatively abnormal light pressure can approximate a lift off event. Accordingly, if the applied pressure changes from a relatively large pressure (whereby the temperature sensors accurately detect actual skin temperature) to a relatively light pressure (whereby temperature sensors will detect a temperature lower than skin temperature) and back to a relatively large pressure (whereby the temperature sensors will again detect the actual skin temperature which is higher than the temperatures previously detected during light pressure), the pressure change may lead to a temperature rise detection similar to zone 21 in FIGS. 4, 5 and result in an erroneous detection of a freeze event. An erroneous freeze event detection can also occur if the applicator moves along the patient's skin to a warmer skin area than the skin area previously being treated because a temperature sensor will then detect a rise in temperature similar to that illustrated in zone 21 in FIGS. 4 and 5. This is because untreated skin (e.g., skin adjacent to a treatment area) is warmer than skin previously contacted by the applicator. For example, an applicator can slide transversely along the skin and a temperature sensor either embedded in the applicator or on its surface can detect an increase in skin temperature. This increase in skin temperature can oftentimes be misinterpreted as a freeze event, when in fact what has occurred is movement of the applicator along the surface of the skin and no freeze event is in fact occurring.

If an erroneous detection of a freeze event occurs, a control module may cause a device to terminate treatment or otherwise send instructions so that cooling by the applicator is terminated or reduced, or initiate other treatment adjustments to prevent or limit or restrict further cooling resulting in the patient being undertreated or premature termination of the procedure. For example, the control module may command the treatment device to stop cooling tissue altogether and terminate a treatment. In addition or in lieu of skin temperature measurements, optical signals, electrical signals, and/or sounds signals (e.g., ultrasound signals) can be used to detect events. A wide range of techniques can be used for either measuring or inferring a frozen or un-frozen state of the tissue, and all of these techniques can result in false detection of freeze events when any movement causes a lift off, a change in pressure, or movement of the applicator.

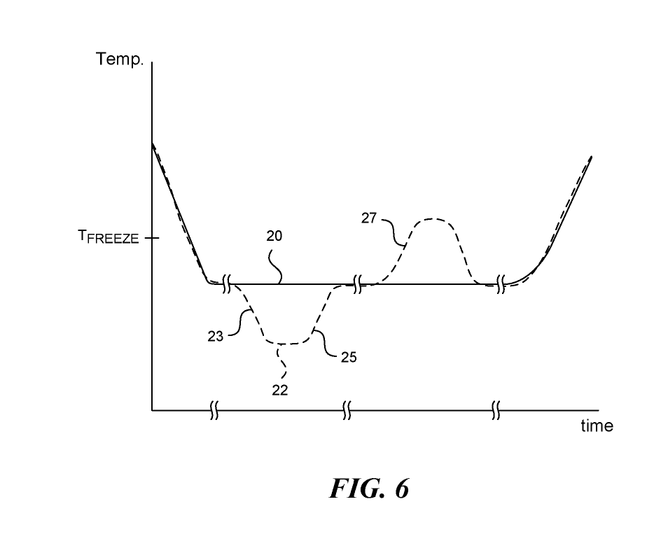

FIG. 6 shows a time versus temperature graph of a representative situation in which the correct skin temperature is shown by solid line curve 20, and the sensed or detected temperature is shown by dashed line curve 22. Zone 23 shows an inaccurate drop in detected skin temperature which as previously explained could be caused by an applicator lift off event or an abnormal light pressure between the applicator and the skin. Upon re-contact with the skin following a lift off event, or a return to normal skin/applicator pressure following a light pressure situation, an inaccurate rise in skin temperature shown by zone 25 can be detected resulting in a false freeze event detection. Alternatively, if the applicator were to move along the skin during a treatment, zone 27 shows an inaccurate detection of a rise in skin temperature which again can be misinterpreted as a freeze event when in fact no skin is freezing. False freeze dashed line zones 25, 27 could be caused by other means other than those just described when noise is detected by the sensors. According to some embodiments of the invention, noise in signals from the sensors and inaccurate treatment skin temperature detections can be minimized, limited and/or filtered out to reduce the difference between the detected and actual skin temperatures, and eliminate and/or minimize false freeze event detections.

Embodiments disclosed herein can address signal noise, false freeze event detection, and other detection problems in several ways. For example, numerous sensor measurements can be taken over time to oversample the measurement. Instead of taking measurements at a low collection rate (e.g., one measurement per second), data collection rates can be continuous or can be relatively high. For example, data collections rates on the order of or in excess of 5, 10, 15, 20, 25, 30, 35, 40, 50, 60, 70, 80, 90, or 100 Hz or higher could be used. The oversampled signal can then be processed in various ways to eliminate noise, read through noise, and/or otherwise reduce noise. For example, sensor signals can be processed to accurately and reliably read through noise and in particular motion generated noise.

In some embodiments, a set of characteristic time verses temperature data can be empirically determined from empirical measurements associated with power levels being used to drive an applicator (e.g., a specific treatment device) and with the treated body part. A set of characteristic time verses temperature graphs can be used to create expected time/temperature templates or other templates used to operate treatment systems. Measured values (e.g., measured temperatures) can then be compared to these templates. An amount of variation between the templates and the measurement values can then be used to estimate an amount of noise in the signal. The larger the noise, the higher the likelihood that the measurements are corrupted with noise and may not be indicative of an actual state of the skin being treated (e.g., measurements may not be indicative of whether or not a freeze event is occurring). When a high amount of noise is estimated, freeze threshold detection values can be adjusted (e.g., increased) to generally suppress false freeze detections.

Additionally or separately, in response to generated noise estimates, signals can be adaptively filtered to eliminate or minimize incorrect data or readings associated with unwanted signal corrupting noise, and to create a filtered signal which can then be analyzed to more accurately determine temperatures or other parameters being measured (e.g., degree of crystallization) and to more accurately determine whether a freeze event is occurring. A wide range of different signal processing techniques can be used to adaptively filter signals from sensors to provide filtered signals. Exemplary techniques include adaptive filtering in general, adaptive low pass filtering, adaptive noise canceling, and/or Kalman filtering, as detailed below.

One example of an adaptive filter according to at least some disclosed embodiments is an adaptive low pass filter. Conventional fixed low pass filters create a filtered signal by averaging a fixed number of consecutive measurements (e.g., 2, 3, or 4 measurements), so as to create a filtered averaged signal. Such conventional fixed filters reject higher frequencies. According to at least some embodiments of the present invention, the number of consecutive measurements averaged can vary, depending on the amount of estimated noise. When the estimated noise is high, the number of measurements can be relatively high, and when the estimated noise is low, the number of measurements can be relatively low. Hence, as the estimated amount of noise varies, a transfer function of the filter can be varied. Adaptively averaging the measurements can result in the transfer function of the filter being continually adjusted in response to the amount of estimated noise. In some embodiments, the estimated noise is derived, at least in part, by comparing signals from sensors to at least one reference value (e.g., a reference signal template) or by measuring a rate of change of the signal and comparing that to prior detected rates of change and/or expected rates of change.

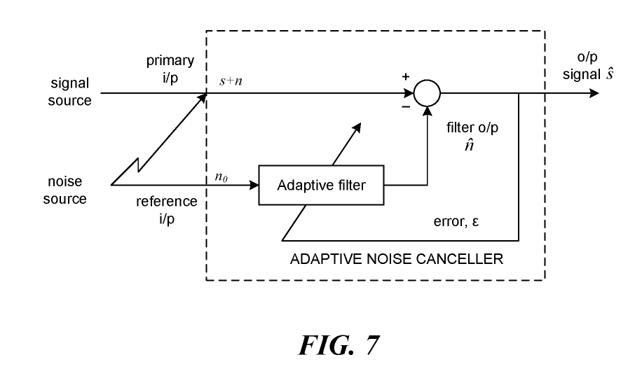

Additionally or separately, a signal from sensors can be adaptively filtered using one or more an adaptive noise cancellers. FIG. 7 shows an adaptive noise canceller (ANC) in accordance with various embodiments of the present invention. The ANC can have a primary input (i/p) and a reference input (i/p). The primary input can receive a signal from the signal source (e.g., sensors 217, 277 of FIG. 3 or other sensors) corrupted by the presence of noise correlated or uncorrelated with the signal. The reference input receives noise nₒ correlated or uncorrelated with the signal and correlated in some way with the noise n. The noise nₒ passes through an adaptive filter to produce an output \hat{n} that may be a close estimate of primary input noise. The noise estimate can be subtracted from the corrupted signal to produce an estimate of the output signal (ŝ) of the ANC system output.

In some noise canceling systems, one objective can be to produce a system output ŝ=s+n−\hat{n} that is a best fit to the signals using least squares technique to signal s. This objective can be accomplished by feeding the system output back to the adaptive filter and adjusting the filter through a least means square adaptive algorithm to minimize total system output power.

The reference input signal can come from many different sources for the primary source used for the signal. U.S. Pat. No. 8,285,390, the disclosure of which is incorporated herein by reference, shows and describes several possibilities for obtaining a primary signal source and a secondary signal source. A signal can be measured on the patient's skin, on a liner attached to a patient, on a surface of a cooling element (cooling plate, patient protection device, etc.) associated with an applicator, in an interior portion of an applicator, or at other locations. The type of signal can also vary. For example, the signal can be an electrical (e.g., impedance, voltage, current) measurement, optical measurement, radiofrequency (RF) measurement, and/or ultrasonic measurement. Mechanical signals or other types of signals from sensors could also be used. For example, accelerometers, vibration sensors, or other types of mechanical sensors capable of detecting motion, vibrations, or the like can be used to provide reference signals.

An array of sensors can provide a single source signal or several source signals for the primary input, and a single source signal or several source signals for the reference input. The types, number, and locations of the sensors can be selected based on, for example, desired monitor/detection capabilities. For example, if a thermistor surface temperature sensor is located on a thermoelectric cooler, additional surface sensors could be spaced from each other. FIG. 3 shows spaced apart sensors 227. Hence, there can be plural measurements which can be used for one or more source primary signals and/or for one or more reference signals. The adaptive noise canceller and algorithms for running the adaptive noise canceller can also vary as a correlation between primary and secondary signal changes depending on the selected source signal, reference signal, and/or other signals. Many ANCs and associated optimum filter algorithm(s) which can be used with the present invention are further described in Adaptive Noise Cancellation, Aarti Singh, 1/ECE/97, Dept. of Electronics & Communication, the disclosure of which is incorporated herein by reference; and in Chapters 6, 12 of Adaptive Signal Processing by Bernard Widrow and Samuel Stearns, published by Prentice Hall, copyright 1985, incorporated herein by reference in its entirety.

Additionally or separately, source signals can be adaptively filtered using a Kalman filter. Kalman filtering can allow parameter fitting using adaptive least squares techniques when parameters vary over time. In contrast to classical least squares techniques with a set amount of averaging, the Kalman filter can calculate an optimal amount of averaging for a desired estimated quantity. At least some embodiments disclosed herein can employ a Kalman filter algorithm and techniques disclosed in R. G. Brown and P. Y. C. Hwang in Introduction to Random Signals and Applied Kalman Filtering (1992), and/or described in U.S. Pat. No. 5,853,364, the disclosures of which are incorporated herein by reference in their entireties. A simplified general Kalman filter usable with at least some embodiments of the present invention is described below.

In this example, an estimate of the data average can be made as data is being measured. The measured data can also have a gain H that to be removed. K-th measurement can be Zₖ and the k-th estimate of the average can be Xₖ. The first estimate of the average can be the measurement.

$${x}_{1} = \frac{{z}_{1}}{H} \qquad ( 9 )$$

After the second measurement, the estimate becomes

$${x}_{2} = \frac{{z}_{1} + {z}_{2}}{2 H} \qquad ( 10 )$$

after the third measurement, the estimate becomes

$${x}_{3} = \frac{{z}_{1} + {z}_{2} + {z}_{3}}{3 H} \qquad ( 11 )$$

This process may be continued. The calculation can become inefficient because of the need to store all of the measurements, constantly re-adding them all, and dividing by the gain and the number of measurements. One efficient solution uses only the last estimate of the average and the current measurement. With this solution, after the first measurement, the estimate is still

$${x}_{1} = \frac{{z}_{1}}{H} \qquad ( 12 )$$

After the second measurement, the estimate becomes

$${x}_{2} = \frac{{x}_{1}}{2} + \frac{{z}_{2}}{2 H} \qquad ( 13 )$$

after the third measurement, the estimate becomes

$${x}_{3} = \frac{2 {x}_{2}}{3} + \frac{{z}_{3}}{3 H} \qquad ( 14 )$$

This approach may be generalized to

$$\begin{matrix}{x}_{k} = ( \frac{k - 1}{k} ) {x}_{k - 1} + \frac{1}{kH} {z}_{k} \\ = {x}_{k - 1} + \frac{1}{kH} ( {z}_{k} - {Hx}_{k - 1} ) \\ = {x}_{k - 1} + K ( {z}_{k} - {Hx}_{k - 1} )\end{matrix} \qquad ( 15 )$$

where K has been used to simplify the equation notation. The Kalman filter uses the same concepts with some extensions: the Kalman filter optimally filters noise, and the parameter being estimated can vary in time.

A simplified Kalman filter employed in one embodiment of the invention will now be described. A parameter to be estimated (for example, temperature) is x which varies in time (e.g., varies in some predictable way). If the value of x is known at some sample in time, then in the next sample, x may be expected to have little or no variation from the previous value. Q can be the variance of this difference. The parameter x is not measured directly. A parameter z is the measured value, which equals x times a constant H plus measurement noise. R is the variance of this measurement noise. Rewriting these

Xₖ=Xₖ₋₁+nₖ^{Q}

Zₖ=HₖXₖ+nₖ^{R}

The ability to estimate the value of x knowing z and the last estimate of x is related to the two noises quantified by R and Q. The Kalman filter can quantify the two noises in a parameter referred to as the estimation error, P. The Kalman filter can also use an intermediate term referred to as the Kalman gain, K. P₀⁻¹ can be initialized with a value of zero. Then at each new data point k, the following acts can be performed:

Pₖ⁻¹=Pₖ₋₁⁻¹+Hₖ²Rₖ⁻¹

Kₖ=PₖHₖRₖ⁻¹

XₖXₖ₋₁+Kₖ(Zₖ−HₖXₖ₋₁)

Pₖ₋₁=Pₖ+Qₖ

The estimate Xₖ looks like the sample-averaging example.

With the Kalman filter, the temperature is allowed to vary, and the model can be separated into two parts. The first part can be:

Vₖ=UₖSₖ+n^{R}ₖ

The ratio of the transformed pre-processed data can be the temperature value except for measurement noise. The spread of the data gives a real-time measurement of the noise variance. The second part shows that on average the temperature does not change in time, but if it does change the standard deviation of the change is generally constant, Q^{1/2}. The second equation can be

Sₖ=Sₖ₋₁+n^{Q}ₖ

This second equation gives the Kalman filter the ability to recognize that if temperature changes by 10° C. in two seconds, for example, it may be due to measurement noise. The Kalman filter then averages the calculated temperature more with previous values to bring the change more in line with what is expected from physiology. In contrast, if the change is within bounds, then the Kalman filter will average very little.

The value of R can be estimated from the difference between V and US over the last N points, where the user specifies the value N. In one embodiment, the Kalman model adds a small incremental value to the actual variance to represent the error inherent in the measurement system (e.g., hardware noise).

The measurement noise can be estimated by centering a window around the data values being used. This centering may give a more accurate estimate of the noise, but may delay the output of the Kalman filter by half the window length. It is believed that a one second window or one half second window may be beneficial because the filter can respond quickly to motion coming and going, and the one-half to one-quarter second delay in temperature estimation may not be clinically significant.

The Kalman filter may behave in a very robust manner. Although motion can fool the Kalman filter, in most instances Kalman filtering results in the calculated temperature remaining closer to actual temperature much longer than the classic least squared (CLS) method and other known non-adaptive methods and adaptive methods.

Since various filter algorithms work differently depending on the type, source and level of noise, at least some embodiments of the present invention can detect freeze events, freeze detection and suppress false freeze alarms by utilizing several different algorithms in parallel on signals, detected data, etc. Hence, treatment systems disclosed herein can utilize one or more fixed filters, including fixed low pass filters and filters using fixed CLS algorithms, and one or more adaptive filters, such as adaptive noise cancellers and Kalman filters. A measurement of noise characteristics of the signals being processed by each filter can be used by each filter to generate a confidence metric. The confidence metric associated with each filter can indicate a likelihood that the measurements are accurate or inaccurate and to what degree and whether or not a freeze detect measurement associated with the signal is correct or not correct. These confidence metrics can then be analyzed to arbitrate between them to best determine or estimate if a freeze event is or is not occurring, and how best to control the treatment system.

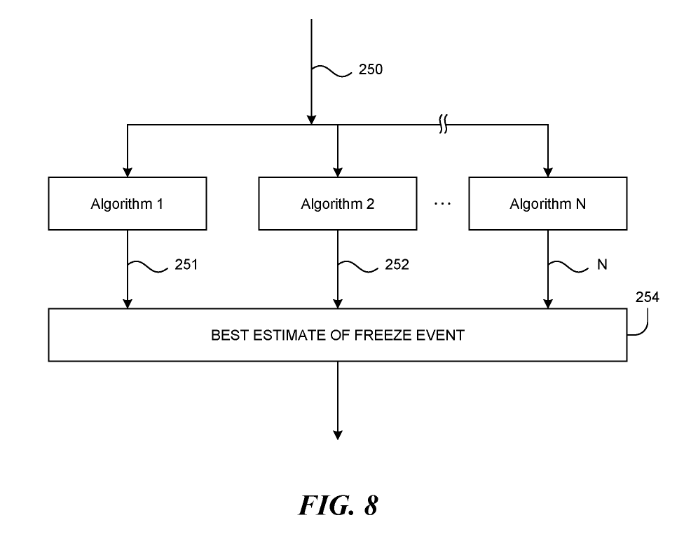

FIG. 8 illustrates a parallel processing embodiment in accordance with the present invention. The signal 250 can be the output from sensors disclosed herein. The signal 250 can be processed in parallel by algorithms 1, 2, . . . , N. An output 251, 252, . . . N of corresponding algorithms can include an estimate of whether a freeze event (or other event) is occurring, a noise estimate (e.g., estimate of noise in the signal 250), and/or a confidence of whether the estimate (i.e., the estimate of whether a freeze event is occurring) is true. A best estimate module 254 can then process all the outputs 251, 252, . . . , N and determine desired information. For example, the best estimate module 254 can determine whether or not a freeze event is occurring, which can then be used by a controller to either turn off a cooling capability of a treatment device, reduce but not turn off a cooling capability of the treatment device, adjust a treatment time of the treatment device, alert a clinician, or take some other action. In some embodiments, algorithms 1, 2, . . . , N output signals to the best estimate module 254, which determines whether adverse treatment-related events will occur or have occurred.

Many of the adaptive filter embodiments described herein can successfully “read through motion,” allowing freeze events to be detected and acted upon in the presence of motion, as opposed to ignoring measurements when significant motion induced signal noise is detected. This enhances detection of freeze events.

### D. Computing Environments

FIG. 9 is a schematic block diagram illustrating subcomponents of a controlling device in accordance with an embodiment of the invention. The controlling device or controller 290 (“controller 290”) can be part of treatment systems disclosed herein. For example, the controller 290 can be the controller 114 of FIG. 1 or can be incorporated into treatment devices disclosed herein. The controller 290 can include, without limitation, a computing device 300 with a processor 301, a memory 302, input/output devices 303, and/or subsystems and other components 304. The computing device 300 can perform a wide variety of computing processing, storage, and/or other functions. The computing processing can include, without limitation, signal processing (e.g., noise reduction, filtering, estimating, etc.), event detection, calibration routines, or the like. Components of the computing device 300 may be housed in a single unit or distributed over multiple, interconnected units (e.g., though a communications network). The components of the computing device 300 can accordingly include local and/or remote memory storage devices and any of a wide variety of computer-readable media. As illustrated in FIG. 9, the processor 301 can be a signal processor programmed to, for example, estimate noise characteristics of signals. Based on estimated noise characteristics, the processor 301 can adaptively filter the signals to provide at least one filtered signal. The computing device 300 can use the filtered signal to determine whether events have occurred or otherwise monitor treatments. In some embodiments, the signal processor 301 can dynamically change its transfer function to adapt to and remove varying amounts of noise from signals 318 based on, for example, the estimated noise characteristic and may include one or more adaptive low pass filters, Kalman filters, and/or adaptive noise cancellers. The adaptive low pass filters can average signals with an amount of averaging being dependent on the noise characteristic estimate. Kalman filters can generate measurements derived from the signals 318. The Kalman filters can compare selected measurements with at least one expected measurement characteristic (e.g., predetermined values) and can assign variable weights to each selected measurement based on the comparing step, thereby generating weighted measurements. The variable weights can be assigned in response to a comparison between each selected measurement and a corresponding previous measurement. The weighted measurements can be averaged to obtain a filtered measurement for use in estimating a likelihood of a partial freeze event. The Kalman filter can selectively average and assign acts, and the adjusting act can be based on knowledge derived independently of the measurements in the generating step, at least one characteristics of the treatment device, tissue, and/or skin. Additionally or alternatively, an adaptive noise canceller can combine signals from multiple sensors to create filtered signals.

In some embodiments, the signal processor 301 is programmed to determine a first likelihood of the freeze event occurring using a first filtering algorithm on the output signal 318, determine a second likelihood of the freeze event occurring using a second filtering algorithm on the output signal 318, and determine whether the freeze event has occurred or is occurring based on the first and second likelihoods. This process can be performed using reference signal templates or other data stored by memory 302. The filtering algorithms can be in the database 310 or stored by memory 302. The algorithms can be used to adaptively filter output signals such that the signal processor 301 dynamically changes to adapt to and remove varying amounts of noise from the signals 318. At least one of the algorithms can be used to evaluate characteristics (e.g., measure a quality) of the signals. Based on quality measurements, the algorithm can be dynamically altered. For example, the transfer function can be altered. Other techniques can be used to process the signals 318.

The signal processor 301 can include functional modules 306, such as software modules, for execution by the processor 301. The various implementations of source code (i.e., in a conventional programming language) can be stored on a computer-readable storage medium or can be embodied on a transmission medium in a carrier wave. The modules 306 of the processor can include an input module 308 (e.g., screen 118), a database module 310, a process module 312, an output module 814, and, optionally, a display module 316.

In operation, the input module 308 accepts an operator input 319 via the one or more input devices, and communicates the accepted information or selections to other components for further processing. The input can be treatment information, event monitoring information, and/or treatment system settings. For example, the operator input 319 can include settings selected to prevent partial freezing, complete freezing, or long freeze events that causes permanent injury to skin.

The database module 310 can organize data (e.g., signal templates, graphs, plots, etc.), recorded signals, records, treatment profiles, patient records, and operating records and other operator activities, and facilitates storing and retrieving of these records to and from a data storage device (e.g., internal memory 302, an external database, etc.). Any type of database organization can be utilized, including a flat file system, hierarchical database, relational database, distributed database, etc.

In the illustrated example, the process module 312 can estimate noise characteristics based on one or more signals (e.g., signals 318 from sensors 277 of FIG. 3). The noise characteristics can be estimated using one or more algorithms stored by memory 302. The process module 312 can generate control variables based on sensor readings 318 and/or other data sources, and the output module 314 can communicate operator input to external computing devices and control variables to the controller. The computing device 300 that modifies operation of the treatment device (e.g., FIG. 1) upon the determination of the event. The display module 316 can display event information, sensor readings 318, or other information useful to the operator of the treatment system 100.

The processor 301 can be a standard central processing unit or a secure processor. Secure processors can be special-purpose processors (e.g., reduced instruction set processor) that can withstand sophisticated attacks that attempt to extract data or programming logic. The secure processors may not have debugging pins that enable an external debugger to monitor the secure processor's execution or registers. In other embodiments, the system may employ a secure field programmable gate array, a smartcard, or other secure devices.

The memory 302 can be standard memory, secure memory, or a combination of both memory types. By employing a secure processor and/or secure memory, the system can ensure that data and instructions are both highly secure and sensitive operations such as decryption are shielded from observation. In various embodiments, the memory 302 can be flash memory, secure serial EEPROM, secure field programmable gate array, or secure application-specific integrated circuit. The memory 302 can store executable instructions for noise processing, causing the applicators to cool/heat tissue, pressurization devices to draw a vacuum, or other acts disclosed herein. In one embodiment, the memory 302 stores instructions executable by the controller (e.g., controller 114) for applicators to sufficiently cool subcutaneous lipid-rich cells to a desired temperature, such as a temperature less than about 0° C. The memory 302 can store algorithms (e.g., correction algorithms, adaptive filtering algorithms, etc.), adaptive noise canceller programs, noise canceling systems, best estimate modules, etc. Additionally or alternatively, measured signals, outputs, calibration routines, filtering routines, or other routines or information can be stored by memory 302.

The input module 308 can include, without limitation, a touchscreen (illustrated as input/output 118 of FIG. 1), a keyboard, a mouse, a stylus, a push button, a switch, a potentiometer, a scanner, an audio component such as a microphone, or any other device suitable for accepting user input and can also include one or more video monitor, a medium reader, an audio device such as a speaker, any combination thereof, and any other device or devices suitable for providing user feedback. For example, if an applicator moves an undesirable amount or lifts off during a treatment session, the input/output device can alert the subject and/or operator via an audible alarm. The operator can reposition the applicator and resume treatment. The input/output device can be a touch screen that functions as both an input device and an output device. The control panel can include visual indicator devices or controls (e.g., indicator lights, numerical displays, etc.) and/or audio indicator devices or controls. The control panel may be a component separate from the input device and/or output device, may be integrated applicators, may be partially integrated with one or more of the devices, may be in another location, and so on. Further details with respect to components and/or operation of applicators, control modules (e.g., treatment units), and other components may be found in commonly-assigned U.S. Patent Publication No. 2008/0287839.

Various embodiments of the invention are described above. It will be appreciated that details set forth above are provided to describe the embodiments in a manner sufficient to enable a person skilled in the relevant art to make and use the disclosed embodiments. Several of the details and advantages, however, may not be necessary to practice some embodiments. Additionally, some well-known structures or functions may not be shown or described in detail, so as to avoid unnecessarily obscuring the relevant description of the various embodiments. Although some embodiments may be within the scope of the invention, they may not be described in detail with respect to the Figures. Furthermore, features, structures, or characteristics of various embodiments may be combined in any suitable manner. Moreover, one skilled in the art will recognize that there are a number of other technologies that could be used to perform functions similar to those described above. While processes or acts are presented in a given order, alternative embodiments may perform the processes or acts in a different order, and some processes or acts may be modified, deleted, and/or moved. The headings provided herein are for convenience only and do not interpret the scope or meaning of the described invention.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.).

Any patents, applications and other references, including any that may be listed in accompanying filing papers, are incorporated herein by reference. Aspects of the described invention can be modified, if necessary, to employ the systems, functions, and concepts of the various references described above to provide yet further embodiments. These and other changes can be made in light of the above Detailed Description. While the above description details certain embodiments and describes the best mode contemplated, no matter how detailed, various changes can be made. Implementation details may vary considerably, while still being encompassed by the invention disclosed herein. Particular terminology used when describing certain features or aspects of the invention should not be taken to imply that the terminology is being redefined herein to be restricted to any specific characteristics, features, or aspects of the invention with which that terminology is associated.

What is claimed is:

1. A non-invasive treatment system for removing heat from a subject's subcutaneous tissue, the treatment system comprising:
a treatment device configured to contact an area of the subject's skin and remove heat from the tissue located below the contacted area of skin;
a first sensor that measures a characteristic of at least one of the treatment device, the tissue, and the skin, the sensor being configured to output a first signal;
a signal processor programmed to estimate a noise characteristic of the first signal and based on the estimated noise characteristic to adaptively filter the first signal to provide at least one filtered first signal, wherein the signal processor is programmed to use the filtered first signal to determine whether at least a partial freeze event has occurred; and
a controlling device that modifies operation of the treatment device upon the determination of the at least partial freeze event.

2. The treatment system of claim 1, wherein the signal processor is programmed to dynamically change its transfer function to adapt and remove varying amounts of noise from the first signal based on the estimated noise characteristic.

3. The treatment system of claim 1, wherein the signal processor includes an adaptive low pass filter, a Kalman filter, and/or an adaptive noise canceller.

4. The treatment system of claim 1, wherein the signal processor includes an adaptive low pass filter which averages the first signal, wherein an amount of averaging is dependent on the estimated noise characteristic.

5. The treatment system of claim 1, wherein the signal processor includes a Kalman filter, wherein the Kalman filter:
generates a plurality of measurements derived from the first signal;
compares selected measurements with at least one expected measurement characteristic;
assigns one of a plurality of variable weights to each selected measurement based on the comparing step, thereby generating a plurality of weighted measurements;
assigns the plurality of variable weights in part, in response to a similarity between each selected measurement and a corresponding previous measurement;
averages the plurality of weighted measurements to obtain a filtered measurement for use in estimating a likelihood of the partial freeze event; and
selectively adjusts at least one step in the averaging and assigning steps, the adjusting step based on knowledge, which is derived independently of the plurality of measurements in the generating step, of at least one characteristic of the treatment device, tissue, or skin.

6. The treatment system of claim 1, further comprising a second sensor which measures a second signal, wherein the signal processor includes an adaptive noise canceller that combines the first and second signals to create the filtered first signal.

7. The treatment system of claim 6, wherein the second sensor is a mechanical sensor, an optical sensor, and/or an impedance sensor.

8. The treatment system of claim 1, wherein the modified operation is selected from the group (a) turning off a cooling capability of the treatment device, (b) reducing but not turning off a cooling capability of the treatment device, (c) adjusting a treatment time of the treatment device, and/or (d) alerting a clinician.

9. The treatment system of claim 1, wherein the signal processor is programmed to determine that the freeze event has occurred by (a) determining when a characteristic of the filtered first signal exceeds a first predetermined value and (b) determining that the first predetermined value is exceeded by a first period of time.

10. The treatment system of claim 1, wherein the first predetermined value and the first period of time are variable and dependent on the estimated noise characteristic.

11. The treatment system of claim 1, wherein the sensor outputs the first signal at a frequency in excess of either 5, 10, 15, 20, 25, 30, 35, 40, 45, or 50 Hz.

12. A non-invasive treatment system for transdermally removing heat from tissue beneath a subject's skin, the treatment system comprising:
a treatment device configured to contact an area of the skin and remove heat from the tissue located below the contacted area of skin;
a first sensor that measures a characteristic of at least one of the treatment device, the tissue, and the skin, wherein the sensor is configured to output a first signal; and
a signal processor programmed to estimate a noise characteristic of the first signal and determine whether at least a partial freeze event has occurred, the determination being based in part on the estimated noise characteristic such that a partial freeze event is determined to not have occurred when the estimated noise characteristic exceeds a predetermined noise characteristic value.

13. The treatment system of claim 12, wherein the estimated noise characteristic is derived in part by comparing the first signal to at least one reference signal template.

14. A method for removing heat from tissue beneath skin and detecting a freeze event in the presence of motion, the method comprising:
a. removing heat from tissue located below a skin surface using a treatment device;
b. measuring a characteristic of at least one of the treatment device, the tissue, and the skin using a sensor, the sensor outputting a first signal;
c. adaptively filtering the first signal to create a filtered signal to eliminate motion artifact and read through motion induced signal noise; and
d. determining whether a freeze event is or has occurred based on analysis of the filtered signal.

15. A non-invasive treatment system for transdermally removing heat from tissue beneath a subject's skin, the treatment system comprising:
a treatment device configured to contact an area of the skin and remove heat from the tissue located below the contacted area of skin;
a sensor that measures a characteristic of at least one of the treatment device, the tissue, and the skin, the sensor configured to output a signal; and
a controlling device that receives the signal and modifies operation of the treatment device upon determining at least a partial freeze event has occurred, the controlling device including a signal processor programmed to:
determine a first likelihood of the freeze event occurring using a first filtering algorithm on the output signal,
determine a second likelihood of the freeze event occurring using a second filtering algorithm on the output signal, and
determine whether the freeze event is occurring based on the first and second likelihoods.

16. The treatment system of claim 15, wherein at least one of the first and second filtering algorithms is used to adaptively filter the signal such that the signal processor dynamically changes its transfer function to adapt to and remove varying amounts of noise from the signal.

17. The treatment system of claim 15, wherein at least one of the first and second filtering algorithms measures a quality of the signal.

18. The treatment system of claim 15, wherein the freeze event includes at least partial freezing of the subject's skin.
